(19) United States
(12) Patent Application Publication
(10) Pub. No.: US 2025/0086234 A1
(43) Pub. Date: Mar. 13, 2025

(54) Surgical System Leveraging Large Language Models

(71) Applicant: OrbSurgical Ltd., Calgary (CA)

(72) Inventors: Homer A. Riva-Cambrin, Calgary (CA); Sanju Lama, Calgary (CA); Rahul Singh, Calgary (CA); Garnette R. Sutherland, Calgary (CA)

(73) Assignee: OrbSurgical Ltd., Calgary, AB (CA)

(21) Appl. No.: 18/465,042

(22) Filed: Sep. 11, 2023

(51) Int. Cl. G06F 16/9032; G06F 16/901; G06F 16/903; G06F 16/9038; G10L 15/183; G10L 15/22; G10L 15/30

(52) U.S. Cl. CPC G06F 16/90332; G06F 16/9024; G06F 16/90335; G06F 16/9038; G10L 15/183; G10L 15/22; G10L 15/30; A61B 18/12; A61B 34/30

(57) ABSTRACT

A natural language request is received for surgical event information associated with one or more network-connected medical devices. Based on the natural language request, a generative artificial intelligence model is prompted to generate a database query. This database query is used to query a graph database which returns, data responsive to the database query. Such responsive information can be conveyed to a user initiating the request in a user interface (e.g., GUI, audio, etc.). In some variations, the results from the graph database are used to poll one or more other models to obtain further contextual information or to provide curation of the results in a more user-friendly and intuitive manner.

### TECHNICAL FIELD

The subject matter described herein relates to machine learning-based techniques for characterizing the use of sensor-equipped surgical instruments and other medical devices to improve patient outcomes.

### BACKGROUND

Medical devices, and in-particular, sensor-equipped surgical instruments and robotic implements are generating increasing amounts of data. However, the ability to readily and timely access desired information about a particular patient, caregiver, medical device or a technique can be limited.

### SUMMARY

In a first aspect, a natural language request is received for surgical event information associated with one or more network-connected medical devices. Based on the natural language request, a generative artificial intelligence (GenAI) model is prompted to generate a database query. A graph database is then queried using the database to obtain data responsive to the query. This data can be provided in various manners (e.g., displayed in a graphical user interface, audibly conveyed through a speaker, transmitted over a computer network to a remote computing device, loaded into memory, stored in physical persistence, etc.).

The GenAI model can take various forms including a large language model and/or an ensemble of models interconnected by an orchestrator.

A memory unit can be used to enhance input to the GenAI model which has a built-in memory center involving long-term and short-term memory.

The natural language request can be a prompt (e.g., user-defined input, etc.) received through a graphical user interface. In addition or in the alternative, the natural language request can be generated by way of verbal instructions through a microphone of a computing device (e.g., phone, tablet, etc.). These verbal instructions can be converted into the natural language request using automatic speech recognition

The natural language request can be used to generate a first prompt which is input into the GenAI model to generate the database query. In some cases, the query can be sanitized prior to it being used to query the graph database.

In some variations, a refined prompt can be generated based on the graph data received from the graph database responsive to the query. This graph data can be used to generate a refined prompt which, in turn, can be input into the GenAI model (or a different model such as a different GenAI model) to generate a second database query. This second database query can then be used to query the graph database and the results of this second database query can be what is ultimately provided.

In some variations, the results of the query of the graph database are inputted into the GenAI model (or a different model such as a different GenAI model). In such cases, the provided data comprises data responsive to the input of the results of graph database into the GenAI model (or different model).

The graph database can be populated in different manners. For example, data streams characterizing use of a plurality of network-enabled medical devices can be monitored by an event streaming platform which generates records in the graph database characterizing events within the data streams.

In an interrelated aspect, a request for surgical event information associated with one or more network-connected medical devices is received. Based on the request, a first machine learning model is prompted to generate a database query. This database query is used to query a database and the results of this query are input into a second machine learning model. The output of the second machine learning model can be provided. In some variations, the first and second machine learning models are different fine-tuned versions of a same large language model (i.e., the models are fine-tuned differently).

In yet another interrelated aspect, a request is received for surgical event information associated with one or more network-connected medical devices. A first large language model is prompted, based on the request, to generate a database query which is used to query a database. Data responsive to the query is input into a second machine learning model and the output of the second large language model can be provided.

The current subject matter is applicable to a wide variety of medical devices including a surgical tool to be manually manipulated by a surgeon as well as a hand-held surgical tool or robotic surgical system to be operated or controlled by a surgeon. In some cases, the network-connected medical devices can comprise one or more physiological sensors characterizing a condition of a patient during a procedure (e.g., a surgical procedure, etc.).

Non-transitory computer program products (i.e., physically embodied computer program products) are also described that store instructions, which when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The current subject matter provides many technical advantages. For example, the current subject matter can, in some variations, be implemented as a one-stop online tool and/or application that is able to provide patient-specific recipes for management or treatment and follow-up guidance through sparse data prompts posed to the tool. Such a tool or application leverages a back-end generative engine enriched with a dynamic repository of information such as contemporary research publications, standards of care principles accepted and practiced or proposed in the field and the like.

In addition, the current subject matter provides an enhanced user interface for accessing both real-time and historical data associated with a medical device such as a sensor-equipped surgical tool or a robotic implement. In addition, the current subject matter provides enhanced techniques for generating queries of a graph database which, in turn, provides more rapid results while consuming fewer computing resources (e.g., physical persistence, memory, compute, bandwidth, etc.). The current subject matter is also technically advantageous in that it provides high quality data (i.e., contextual data) associated with a particular request, taking advantage from the pool of high ranked clinical publications and guidelines for evidence-based practices in the field.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a system diagram illustrating aspects of a surgical system leveraging large language models;

FIG. 2 is a diagram illustrating the relationship amongst data stored in a graph database;

FIG. 3 is a diagram illustrating a schema of a graph database;

FIG. 4 is a system diagram illustrating a data integration process for use with a graph database.

FIG. 5 is a diagram illustrating aspects of a graph consumer;

FIG. 6 illustrates an JSON model overlaid on objects implied in the schema of a graph database;

FIG. 7 is a diagram for generating curated responses from a graph database using a single model;

FIG. 8 is a diagram for generating curated responses from a graph database using two different models; and

FIG. 9 is a process flow diagram for providing surgical related information using one or more large language models.

### DETAILED DESCRIPTION

The current subject matter is directed to a computing architecture for providing more rapid and contextual information associated with the real-time, near real-time, and/or historical use of network-connected medical devices. This computing architecture is sometimes referred to herein as an intelligent surgical systems. This intelligent surgical system, in turn, provides enhanced techniques and systems for monitoring or otherwise characterizing use of a surgical instrument during one or more surgical procedures. While the current subject matter is described, as an example, in connection with sensor-equipped forceps and robotic implements, it will be appreciated that the current subject matter can also be used with other network-connected medical devices including sensor-equipped surgical instruments including, electrosurgical (bipolar or monopolar) or otherwise, without limitation, cutting instruments, grasping instruments, and/or retractors.

As noted above, the current subject matter can be used with sensor-equipped forceps such as that described in U.S. Pat. Pub. No. 20150005768A1 and entitled: “Bipolar Forceps with Force Measurement”, the contents of which are hereby incorporated by reference. The surgical instruments used herein can include one or more sensors such as an identification sensor (e.g., RFID, etc.), force sensors, motion sensors, position sensors. The data generated from such sensors can be connected to a developed signal conditioning unit interfaced through a software with machine learning algorithm (federated and global) deployed to the cloud (or in some cases executing at a local endpoint). The machine learning algorithms can interface with a unique federated learning architecture such that tool, sensor and surgeon specific data, are recognized, segmented and analyzed (signal, task, skill, pattern (position, orientation, force profile)-all based on sensor signal), such that high fidelity feedback can be generated and provided in real-time (warning) or performance reporting (via secure application or online user profile).

Of note, in our particular environment and market, our intelligent devices installed and operating across the globe (IoI-OR), in real-time, receives, analyzes, outputs and streams meaningful data queried by users across the globe. In so doing, the machine learning is perpetually enriched and trained to continually improve its output, feedback and response, such that surgical decision making is greatly enabled and guided, much like a modern sensor-laden car enabling the driver with confidence, safety and security. In addition, there is a built in federated learning (AI) for international data security and compliance, and a built-in memory for perpetually refined intelligent response to query.

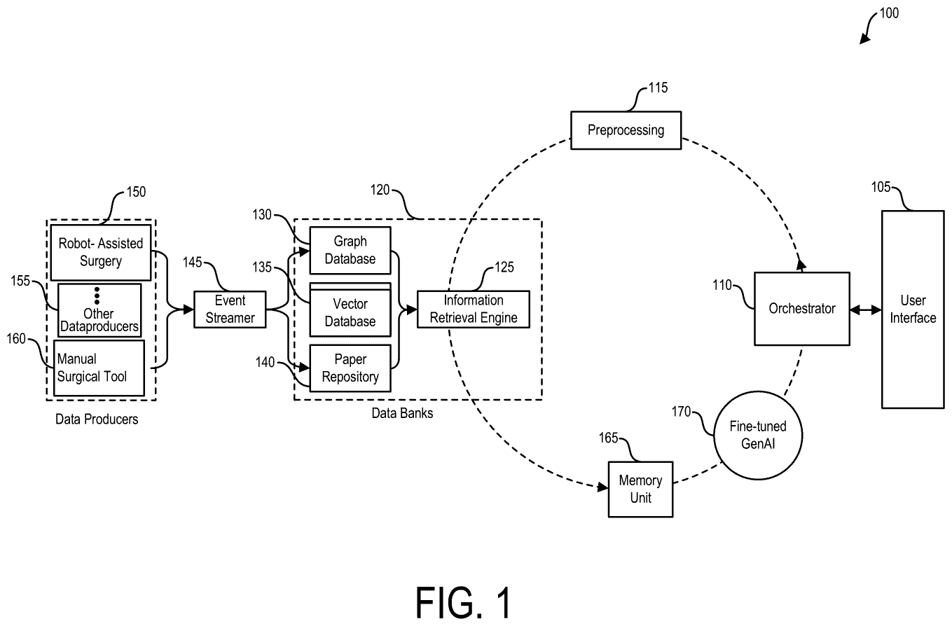

FIG. 1 is diagram 100 of a sample workflow for implementing aspects of the current subject matter. A request is received by way of a user interface (UI) 105. This request can be, for example, one or more alphanumeric strings entered through a graphical user interface (GUI) prompt (e.g., a browser, etc.). In addition or in the alternative, the UI 105 can comprise a voice interface in which the user provides oral information which is then translated into one or more alphanumeric strings using, for example, automatic speech recognition (ASR).

The UI 105 is in communication with an orchestrator 110 which can receive the requests (e.g., the alphanumeric strings) from the UI 105 and which can also provide information (as will be described in further detail below) to the UI 105 responsive to requests. This information can be rendered in the GUI and/or conveyed orally (i.e., there can be an audio component to the information provided in response to a request). The orchestrator 110 after receiving a request can cause the request to be ingested by a preprocessing module 115. The preprocessing module 115 can comprise one or more machine learning models which are trained and configured to take alphanumeric strings (such as those provided through the UI 105) into a query. In some examples, the preprocessing module 115 includes one or more transformer models.

The transformer models can be configured to pre-process a lengthy question trained on synthetic data which can be configured to transform poorly worded natural language questions into specific natural language questions. These transformer models can also be configured (through training using synthetic data) so as to turn conversational context into a set of specific natural language queries. The synthetic data training process for configuring the transformer models can be utilize a large language model and train a smaller instruction model using transfer learning. As an example, Vicuna-13B-v1.5 (which is a fine tuned LLaMa2) can be used to generate text that was then used to train Long T5.

The preprocessing module 115 is configured to transmit a query to a plurality of data banks (DB) 120 (graph database 130, vector database 135, paper repository 140) which includes an information retrieval engine 125. The graph database 130 (sometimes referred to as a knowledge graph), e.g. Neo4j, is a database wherein the database defines objects as nodes and direct relationships between these individual nodes, i.e. the relationship is at the node level; a common alias to graph DB being knowledge graph. For example, a surgeon could have a relationship with a hospital he works at, and another surgeon might not have that relation because he is unemployed. Since the relation is at the record (node) level, unique relationships can exist on each node. The vector database 135 can store high dimensional vectors; as AI can convert text into embedding vectors, which capture semantic meaning of the text, implying searches can be created and executed on semantic meaning. The paper repository 140 wherein the database will have all the publications entered (manuscripts, textbook chapters, patents etc.).

The information retrieval engine 125 can query various data sources 130, 135, 140 for information responsive to the query. The information retrieval engine 125 retrieves and combines data from several sources of information (which can form part of the data banks 120) that is relevant to the query using semantic search methods. One of data sources can comprise a knowledge graph 130 which will be described in more detail below (and also sometimes referred to as a graph database). Other data sources 135 can be queried by the information retrieval engine 125 for information responsive to the query including as well as a paper repository 140 (i.e., a repository of scientific publications, etc.). All of these data sources 130, 135, 140 are queried by the information retrieval engine 115. For instance, a surgeon might ask a question relative to a specific surgical procedure, and/or relevant pathological diagnosis, for which proposed further treatment or follow-up paradigm would be enhanced not only by the particular surgeon's knowledge, expertise and experience on the disease as practiced standard of care, but with added input generated by the information retrieval engine 115 that relies both on relevant wealth of up-to-date information from a built-in semantic search engine and a relational database (thus eliminating the need for accessing multiple contemporary online databases such as Pubmed/medline/WebMD/Recent Updates) saving time and increasing reliance.

The results obtained by the information retrieval engine 125 can be input into a generative AI (GenAI) model 170 (e.g., a fine-tuned large language model configured for caregivers seeking information from various surgical and medical device technologies, etc.) which then generates an output which is conveyed to the UI 105 by way of the orchestrator 110. In some cases, a memory unit 165 provides additional contextual information about the request (e.g., information about a preceding and related request, information about the requesting user in cases of conversational context, etc.).

The data sources 130-140, and in particular, the knowledge graph 130 can be populated using data provided by an event streamer 145. The event streamer 145 can, for example, comprise an event streaming platform (e.g., KAFKA, etc.) and can, as described in further detail below, obtain event data from one or more medical devices including a robotic implement 150, other network-connected medical devices 155, and a network-connected surgical tool 160.

The system of FIG. 1 can be installed and operated across the globe (IoI-OR), and in real-time, receive, analyze, output and stream meaningful data queried by users. In so doing, the machine learning is perpetually enriched and trained to continually improve its output, feedback and response, such that surgical decision making is greatly enabled and guided. In addition, federated learning can be used, for example, for purposes such as international data security and compliance. One technique for federated learning is described in U.S. Pat. No. 11,276,001, the contents of which are hereby fully incorporated by reference. A built-in memory unit 165 can also be used for providing perpetually refined intelligent responses to query.

### Graph Database

As noted above, the current system can utilize a graph database 130. The graph database 130, especially in the surgical theatre setting, provides advantages over conventional techniques (e.g., relational table databases or SQL, etc.) including: providing a truer representation of the data (schema is more appropriate), providing much faster results for complex and simple queries, and representing the data in a manner for a large language model (LLM) which, in turn, allows for integration with a clinically relevant Q&A system.

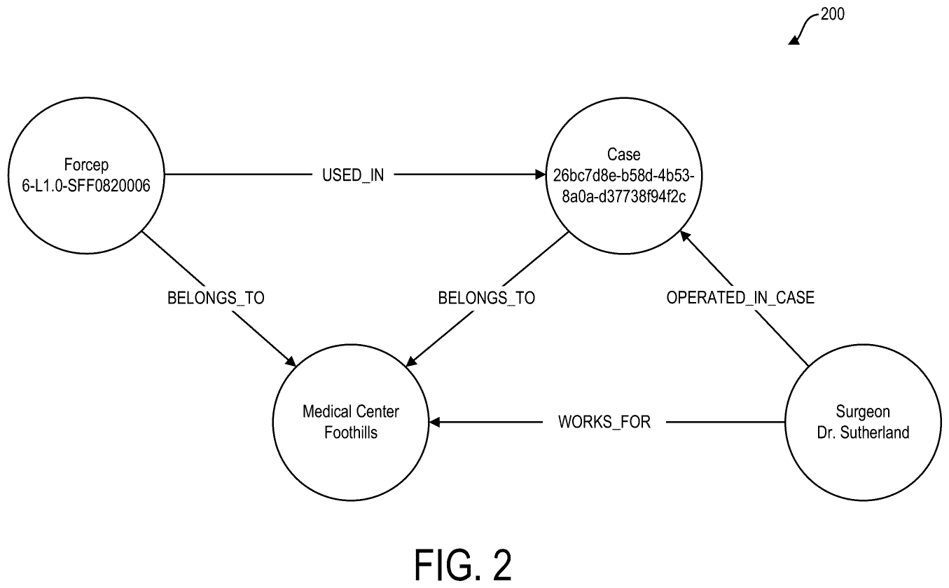

The graph database 130 (sometimes referred to as a knowledge graph), is a way to represent data relationally. The graph database 130 stores data in a manner in which there are nodes which have one or more types and one or more properties which can be connected to other nodes via a relationship. FIG. 2 is a diagram 200 illustrating a simplified extract of data that can be stored within the graph database which illustrates an interrelationship among a medical device (e.g., forceps, etc.), how that medical device has been used (e.g., case, etc.), where the medical devices was used (e.g., medical center, and the associated caregiver (e.g., surgeon, etc.). It will be appreciated that other contextual information can be stored in the graph database 130 including information about other medical devices concurrently used for a patient and/or by a caregiver and their relationship. Such an arrangement is particularly advantageous for use by an intermediate LLM (e.g., the GenAI model 170) to generate a query in response to a request as provided herein.

With the data model for the graph database 130, all data is modeled relationally, which is similar to the manner in which humans think. With reference to FIG. 2, consider all the things can be easily described from this model, for example, “Dr. Sutherland, who works for Foothills, operated on a case, where forceps 6-L1.0-SFF08200006 was used.”

The data stored in the graph database 130 can be queried by the information retrieval engine 125 in a query language (e.g., ‘CYPHER’, etc.) which tells the graph database 130 how to perform operations on the graph. The query language can be used to create new records in the knowledge graph, or to simply query existing data within the knowledge graph. The query language can be written to be highly relational and allow for advanced logic to be embedded in queries. As will be described below, this query language can provide a basis to integrate one or more AI models.

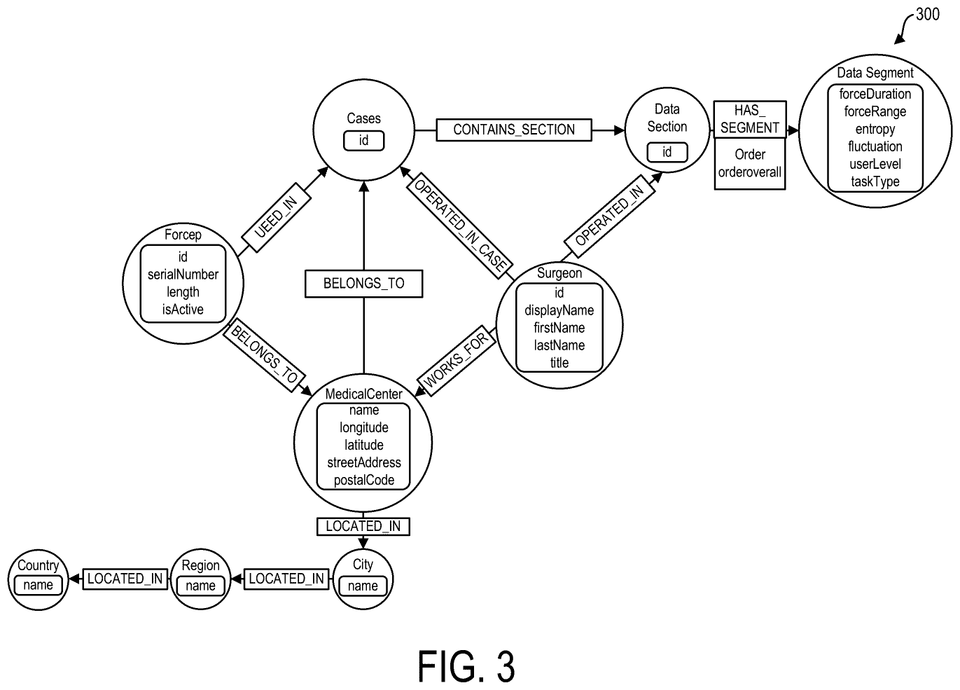

FIG. 3 is a diagram 300 which illustrates an example schema for the graph database. This schema was designed to enable relevant common and/or sufficient direct queries, without putting in an excessive number of connections causing slowdowns. An example of this is the forceps node. They can be used in cases and can belong to a medical center. This enables a multitude of questions: “Which forceps belong to Foothills?” “Which medical center do these forceps belong to?” “Which forceps were used in this case?” However, this also exposes some implicit relationships. For instance, if one wants to know which forceps a certain surgeon has used before, the graph database can be used to identify the cases a surgeon has operated in, and then the forceps used in those cases.

A highly relational query language such as CYPHER is not only user-friendly but it is quite fast as well. Queries based on this language can be easily generated, as provided herein, by the artificial intelligence models such as LLMs or model ensembles including LLMs forming part of the preprocessing module 115. Another design consideration of the schema is malleability so that the schema is easy to change and adapt. This type of adaptability and extensibility can be particularly helpful as new medical devices and/or other features are extracted and stored in the graph database 130.

### Data Loading Procedure

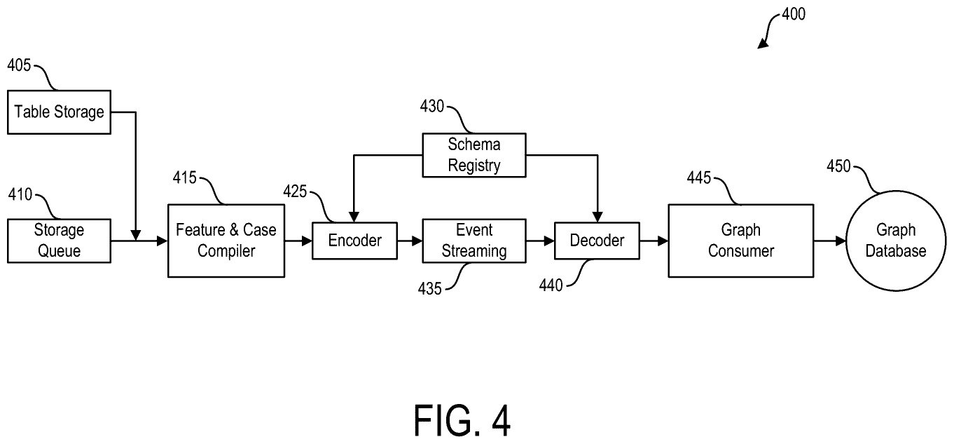

For systems to interact and interface with the data in the graph database 130, new cases need to be successfully integrated into the graph database 130. An overview of an example procedure is illustrated in diagram 400 of FIG. 4. Illustrated is a mechanism for integrating this data, namely a directive 405, 410 placed in a queue, here 405 and 410. In other variations, an event-driven approach can be utilized which live streams data. The directive has a selector that tells a feature and case compiler 415 to pull either the case, forceps, surgeon, and data section records or the feature records. The feature records can be stored in the table storage 405 and, in some cases, they are not available in real-time, whereas in other cases, they may be available in real-time via query (e.g., in-memory, etc.).

The graph database compiler 415 pulls records from either a tabular storage database 405 or from a storage queue 410 in order to convert them into a meaningful output. The storage queue 410 can serve as a trigger prompting the compiler 415 to retrieve relevant information from the tabular storage database 405. This feature avoids the cost and need to continually run a compiler script 24/7.

An encoder 425 encodes the records to make sure that the data is more compact and can be sent through the event streaming platform 435 more efficiently. This encoding can be done using a system called AVRO which encodes the data based on a schema having an associated schema registry 430. The schema registry 430 allows for adding fields and adapting the data being sent as time goes on, enabling better modularity and scalability. A decoder 440 and graph consumer 445 decodes the data from the Event Streaming Platform 435 using the AVRO schema pulled from the schema registry 430 and puts it in the graph database 450.

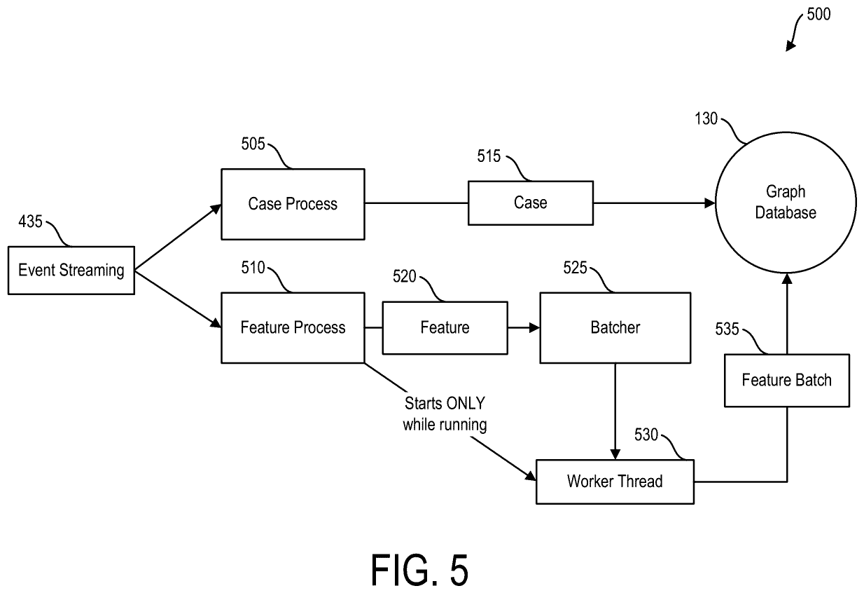

The graph consumer 445 plays an integral part of the event streaming system 435. FIG. 5 is a diagram 500 illustrating how the graph consumer works internally. Within the graph consumer 445, there are two event consumers 505, 510 that respectively listen for events associated with cases and features. The case process 505 is deals with all the data that is made immediately available when the cases are inserted in the tables in table storage 405.

AVRO works off the JSON format and converts it into a binary form. This arrangement provides a guarantee that this data is encoded in the schema which obviates the need to send the schema with the data every time (thereby reducing data transmission cost and increasing transmission speed). The data is received by the case process 505 and is turned into a set of CYPHER queries passed to the graph database 130 to create several relationships and nodes. As an example, the graph database 130 can create surgeon, forceps, data sections, medical center, country, region, city, case nodes and properties, create relationships between all these nodes, and provide for linked AI with built-in memory (forming part of memory unit 165) that connects this info and generates an intelligent, accurate and data-driven response.

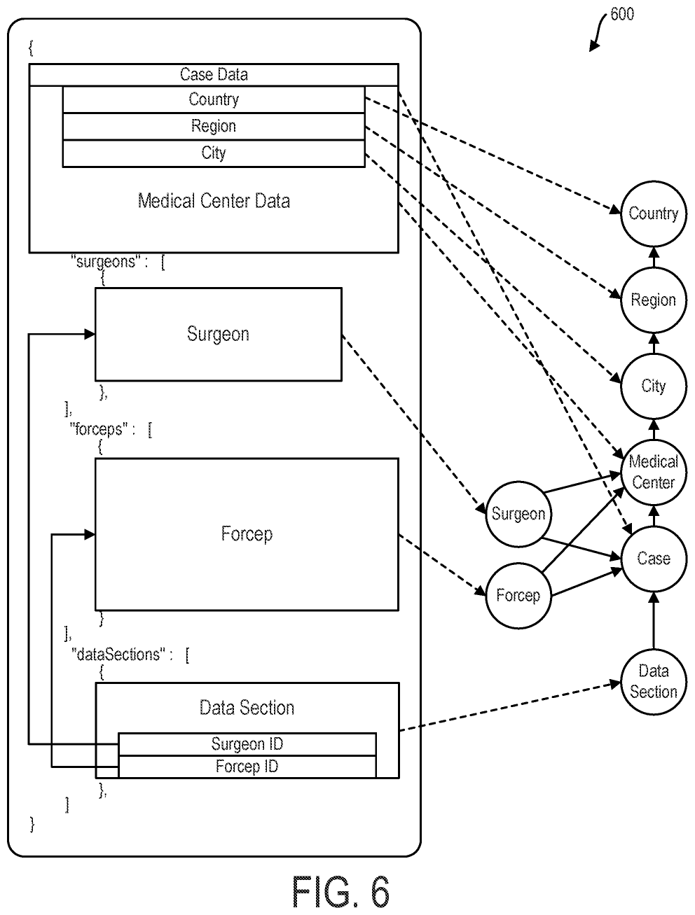

The case schema can be configured to compact the data as it is modeled as a set of related objects to reduce repetition. Diagram 600 of FIG. 6 illustrates how this is modeled conceptually and how it is translated into the knowledge graph.

The next component of the graph consumer 445 is the heavy throughput data, namely the features 520. A case 515 could have several tens of thousands of this type of data. The graph consumer 445 works by receiving many records asynchronously with a schema which allows for the creation of a data segment in the graph database 130 which contains, for example, the properties fluctuation, entropy, force duration, force range, user level, and finally task type. These records are taken by batched using a batcher 525. The batch settings are around ˜10,000, and these are then sent to a worker thread 5430 which sends them out (as a feature batch 535) in the background. This allows for enormous amounts of data to be processed at the same time and to placed into the graph database 130.

### Event Streaming

With the event streaming system 145 there is a broker, producers (which push data to the broker) and consumers (which receive data). The core benefits provided by the event streaming system 145 including high throughput and quick handling of data from many producers and many consumers thereby enabling large number of inputs and subscribers that can read and interact with that data. Further, a topic system (which is where messages are inserted and which can include a case topic and a feature topic) allows data to be organized in a very logical way. The event streaming system 145 also provides high durability and safety as event streaming has a focus on replication and data persistence (meaning it is suitable for mission critical systems). Lastly, the event streaming system 145 is advantageous in that it is exceedingly scalable which is important as the use of network-connected medical devices continues to grow.

### Orchestrator

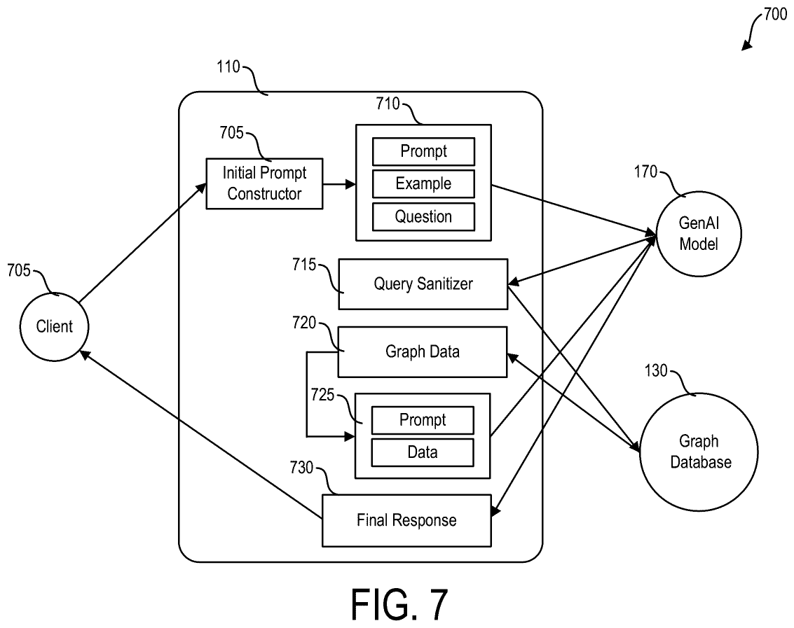

FIG. 7 is diagram 700 illustrating aspects of the orchestrator 110 in which a request received from a client 705 (for example through the UI 105) is used to construct an initial prompt 705. The initial prompt can for example include a prompt that additionally includes an associated example and question. The GenAI model 170 (e.g., GPT, etc.) then returns a result which can be sanitized by a query sanitizer 715 which can act to remove incorrect or malicious aspects from a generated query. Once sanitized, the resulting query can be used to poll the graph database 130 to result in graph data. In some cases, the graph data can be used to generate a subsequent prompt 725 which is used to poll the GenAI model 170 resulting in a final response 730 which can be conveyed to the user by way of the UI 105 executing on the client 705.

The prompt 710 is generated, in real time, upon query by the orchestrator 110 and can include a graph schema. As noted above, the prompt 710 can give a set of examples, finally with a directive and a question which tells the GenAI model 170 (e.g., LLM, etc.), for example, to generate a CYPHER query. The directive can use some prompt engineering techniques to improve the quality of the response:
“Let's work this out in a step-by-step way to be sure we have the right answer. Generate CYPHER statement from the possible parameters and relationships above to query the database without explanation.”

Since the GenAI model 170 may not be fine-tuned there is a possibility for the model to generate odd output such as ‘This data is not relevant enough to generate a query.’ In this case, that will never be handed to the graph (which would cause an error) and will be replaced with a text that says:
“I am a medical professional who asked QUESTION which was not worded in a clear enough way to meaningfully query a graph database with the following schema: SCHEMA. Tell me my question is too broad/not clear enough, and, without mentioning the graph or graph database, based on the graph give me some suggestions on how I can improve the question to get more specific results.”

This text causes the GenAI model 170 to respond with some very useful output, telling the user that their question is not specific enough and giving them some useful suggestions, although sometimes it may provide questions that are not relevant. If the output is solid, it is used to query the graph and then it is handed to the AI with a separate prompt:
“You are a helpful and explanative AI that can interpret data. In this scenario, I (a medical professional) have asked QUESTION which was turned into a query to a graph database. This graph database is about surgeries done with a medical device that includes a sensor that records force data when used in cases. It returned the following data: OUTPUT DATA. Answer by showing and interpreting the data in a medical context.”

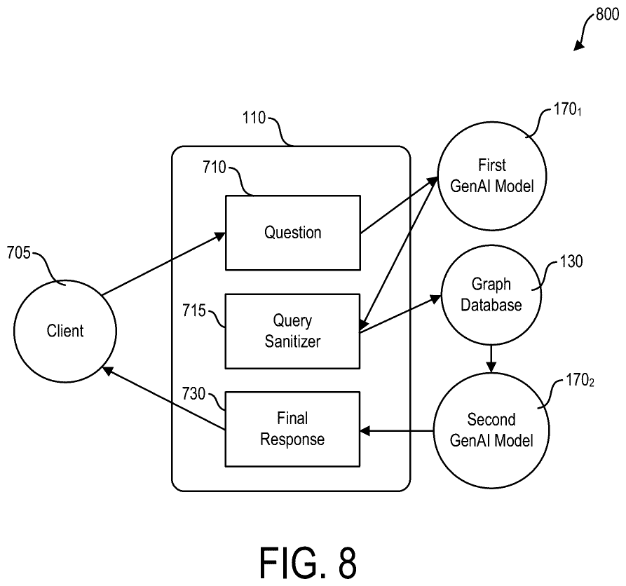

The strength of the GenAI model 170 comes from the fact that graph data is inherently relational like language. As a result, the GenAI model 170 can interact with it via CYPHER to ask it questions. This data can then be given to the GenAI model 170 to draw powerful inferences from the data. While the previously examples provided for a single GenAI model 170, in some variations different models can be used. With reference to diagram 800 of FIG. 8, the system can include a first GenAI model 170/can can be configured for query generation and a second GenAI model 1702 can be configured to generate responses. This bifurcation, in some cases, can allow the first GenAI model 170/to generate higher quality queries and therefore pull more advanced data from the graph database 130 as opposed to a combined approach. Further, these two GenAI models 1701, 1702 can be fine-tuned for their particular application. Such an arrangement also reduces compute costs through lowering of the token count, as this arrangement does not need to ship all the extra text that makes up the prompt to a fine-tuned model.

### GenAI Model

The GenAI model 170 is a model that takes input and generates output based on that input and which can be fine-tuned for the current application. This output can, for example, be recommendation or data-driven predictions based on pre-trained clinical data from the device, surgeons/clinicians, and publications. For example, the GenAI model 170 can be fine-tuned using real answers from surgeons mixed with model-generated answers (which have been deemed accurate by a human supervisor). The GenAI model 170 can take different forms including a large language model such as, but not limited to, Cohere, Claude, Vicuna, and LLaMa.

### Memory Unit

The memory unit 165 is configured to reflect human memory processes whereby the model orchestrator 110 is able to automatically direct query to the most relevant built in information context in storage (the memory center), to appropriately retrieve information from the storage (the memory center) and present memory-enriched relevant responses. The memory unit 165 can comprise a system to hold and retain conversational context in a way that can be loaded into memory and stored in physical persistence (e.g., disk). The memory unit 165 can also utilized an algorithm using transformer models to highlight semantic relevance of sentences and sentence chunks. In some cases, the memory unit 165 can retrieve using, for example, a retrieval algorithm, the most relevant chunks to continue the conversation in addition to chunks that would be passed on and held in memory. The memory unit 165 can also include a transformer model configured to convert the chunks into useful context for a larger language model. The memory mechanism herein can use a variant of semantic vector embeddings in order to generate a heat map over tokens, e.g.-text, in order to retrieve the more relevant segments, akin to large scale attention mechanism.

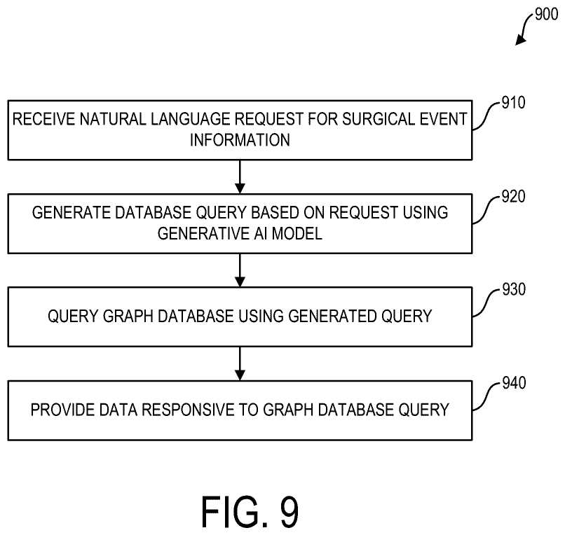

FIG. 9 is a process flow diagram 900 in which, at 910, a natural language request is received for surgical event information associated with one or more network-connected medical devices. Based on the natural language request, at 920, a generative artificial intelligence model is prompted to generate a database query. This database query is used, at 930, to query a graph database which returns, at 940, data responsive to the database query. Such responsive information can be conveyed to a user initiating the request in a user interface (e.g., GUI, audio, etc.). In some variations, the results from the graph database are used to poll one or more other models to obtain further contextual information or to provide curation of the results in a more user-friendly and intuitive manner.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, solid state drives, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein do not require the particular order shown, or sequential order, to achieve desirable results. Other embodiments may be within the scope of the following claims.

1. A computer-implemented method comprising:
receiving a natural language request for surgical event information associated with one or more network-connected medical devices;
prompting, based on the natural language request and with a directive and question to generate a database query, a generative artificial intelligence (GenAI) model to generate the database query, the database query being in a relational query language;
querying a graph database using the database query, the graph database being separate and distinct from the GenAI model; and
providing data responsive to the database query.

2. The method of claim 1, wherein the GenAI model is a large language model.

3. The method of claim 1, wherein the GenAI model comprises multiple models interconnected by an orchestrator.

4. The method of claim 1, wherein a memory unit is used to enhance input to the GenAI model, the memory unit having a built-in memory center involving long-term and short-term memory.

5. The method of claim 1, wherein the natural language request is a prompt received through a graphical user interface.

6. The method of claim 1 further comprising:
receiving verbal instructions to generate the natural language request; and
converting the verbal instructions into the natural language request using automatic speech recognition

7. The method of claim 1 further comprising:
generating a first prompt based on the natural language request, the first prompt being input into the GenAI model to generate the database query.

8. The method of claim 1 further comprising: sanitizing the query generated by the GenAI model prior to querying the graph database.

9. The method of claim 1, wherein the graph database returns graph data in response to the generated query and the method further comprises:
generating, based on the returned graph data, a refined prompt;
inputting the refined prompt into the GenAI model to generate a second database query; and
querying the graph database using the second database query, wherein the provided data responsive to the database query comprises data responsive to the second database query.

10. The method of claim 1, wherein the graph database returns graph data in response to the generated query and the method further comprises:
generating, based on the returned graph data, a refined prompt;
inputting the refined prompt into a second, different GenAI model to generate a second database query; and
querying the graph database using the second database query, wherein the provided data responsive to the database query comprises data responsive to the second database query.

11. The method of claim 1, wherein the graph database returns graph data in response to the generated query and the method further comprises:
generating, refined database queries based on the returned graph data;
inputting the queries to the database to generate the output;
the output are automatically prompted into a second, different GenAI model fine-tuned to generate a meaningful and relevant response in relation to the query and/or the surgical event information.

12. The method of claim 1 further comprising:
inputting results of the database query into the GenAI model;
wherein the provided data comprises data responsive to the input of the results of graph database into the GenAI model.

13. The method of claim 1 further comprising:
inputting results of the graph database into a second, different GenAI model;
wherein the provided data comprises data responsive to the input of the results of graph database into the GenAI model.

14. The method of claim 1 further comprising:
monitoring streams characterizing use of a plurality of network-enabled medical devices;
generating, by an event streaming platform and based on the monitored streams, records in the graph database.

15. The method of claim 1, wherein the one or more network-connected medical devices comprises: a surgical tool to be manually manipulated by a surgeon.

16. The method of claim 1, wherein the one or more network-connected medical devices comprises: a hand-held surgical tool or robotic surgical system to be operated or controlled by a surgeon.

17. The method of claim 1, wherein the one or more network-connected medical devices comprises a physiological sensor.

18. The method of claim 1, wherein the providing data comprises one or more of: causing the data to be displayed in a graphical user interface, causing the data to be audibly conveyed to a user, transmitting the data over a computing network to a remote computing device, loading the data into memory, or storing the data in physical persistence.

19. A computer-implemented method comprising:
receiving a request for surgical event information associated with one or more network-connected medical devices;
prompting, based on the request and with a directive and question to generate a database query, a first machine learning model to generate a database query, the database query being in a relational query language, the first machine learning model being fine-tuned to generate database queries;
querying a database using the database query, the database being separate and distinct from the first machine learning model;
inputting data responsive to the database query into a second machine learning model, the second machine learning model being separate and distinct from the database and the first machine learning model, the second machine learning model being fine-tuned to provide information relevant to surgical event information associated with the one or more network-connected medical devices; and
providing the output of the second machine learning model.

20. The method of claim 19, wherein the first machine learning model and the second machine learning model comprise different fine-tuned models.

21. A computer-implemented method comprising:
receiving a request for surgical event information associated with one or more network-connected medical devices, the one or more network-connected medical devices comprising a sensor-equipped surgical instrument;
prompting, based on the request and with a directive and question to generate a database query, a large language model to generate a database query, the database query being in a relational query language;
sanitizing the database query to remove incorrect or malicious aspects from the generated database query:
querying a database using the sanitized database query, the database being separate and distinct from the large language model;
inputting data responsive to the database query into the machine learning model; and
providing the output of the large language model.
